(19) United States
(12) Patent Application Publication
(10) Pub. No.: US 2019/0185417 A1
(43) Pub. Date: Jun. 20, 2019

(54) NOVEL ANTIBIOTICS

(71) Applicant: Boulos & Cooper Pharmaceuticals Pty Ltd, Balcatta, Western Australia (AU)

(72) Inventor: Ramiz BOULOS, Yokine (AU)

(21) Appl. No.: 16/309,431

(22) PCT Filed: Jun. 27, 2017
(86) PCT No.: PCT/AU2017/050654

§ 371 Date: Dec. 12, 2018

(30) Foreign Application Priority Data
Jul. 1, 2016 (AU) 2016902604

(51) Int. Cl. C07C 237/20; A61P 31/04; C07C 211/27; C07C 205/06; C07C 211/50; C07C 211/63; C07C 229/34; C07C 233/65; C07C 233/11; C07C 239/22; C07C 255/51; C07D 213/53

(52) U.S. Cl. CPC C07C 237/20; A61P 31/04; C07C 211/27; C07C 205/06; C07C 211/50; C07D 213/53; C07C 229/34; C07C 233/65; C07C 233/11; C07C 239/22; C07C 255/51; C07C 211/63

(57) ABSTRACT

Novel aryl compounds or pharmaceutically acceptable salts thereof, and uses of the same for the treatment of Gram-negative infections.

### TECHNICAL FIELD

The present invention relates to novel aryl compounds. More particularly the present invention relates to novel aryl compounds and their use as antimicrobials to treat bacterial infections or diseases caused by Gram-negative bacteria.

### BACKGROUND ART

Compounds with antimicrobial properties have attracted great interest in recent times as a result of an increase in the prevalence of infections caused by Gram-negative bacteria, resulting in serious or fatal diseases. There has also been an increase in the occurrence of bacterial strains resistant to some antimicrobial formulations.

Novel antimicrobial compounds have the potential to be highly effective against these types of treatment-resistant bacteria. The pathogens, having not previously been exposed to the antimicrobial formulation, may have little to no resistance to the treatment.

The above discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides compounds of Formula (I):

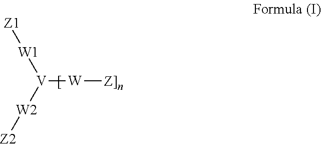

wherein;
V is covalently bonded to W;
W is covalently bonded to Z;
V is a six membered, aromatic ring;
n is 0,1, 2, 3, or 4;
the, or each, W is independently selected from the group;
C₂₋₄ alkyl groups; C₂₋₄ substituted alkyl groups; C₂ E-alkene, with V and Z in the 1 and 2 positions; C₂ Z-alkene, with V and Z in the 1 and 2 positions; C₂-alkyne;
the, or each, Z is independently selected from the group;

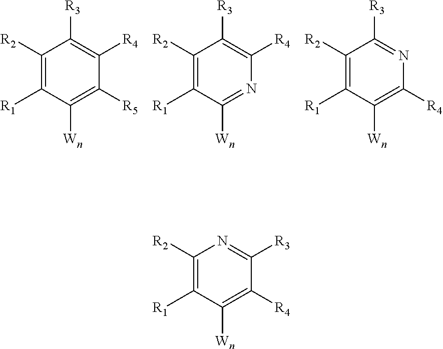

wherein Wₙ is one of W1, W2 or the or each W;
and wherein;
at least one of R₁, R₂, R₃, R₄ or R₅, on at least one of W1, W2 and the or each W is selected from the group;
alkylphosphines of the formula

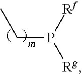

alkyl amines of the formula

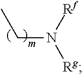

wherein each of R^{f} and R^{g} is independently selected from;

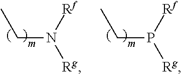

H, C₁-C₈ alkyl; and m is 1-8;
and wherein;
each of the remaining R₁, R₂, R₃, R₄ or R₅ is independently selected from;
halogen selected from the group F, Cl, Br, I; C₁₋₈ alkyl; carboxylic acid of the formula —C(O)OH; alkyl carboxylic acid of the formula —(CH₂)ₙC(O)OH; thiocarboxylic acid of the formula —C(S)OH; Alkyl thiocarboxylic acid of the formula —(CH₂)ₙC(S)OH; ester of the formula —C(O)OR^{a}; alkyl ester of the formula —(CH₂)ₙC(O)OR^{a}; Thioester of the formula —C(S)OR^{a}; alkyl thioester of the formula —(CH₂)ₙC(S)OR^{a}; dithioester of the formula —C(S)SR^{a}; alkyl dithioester of the formula —(CH₂)ₙC(S)SR^{a}; amide of the formula —C(O)NR^{a}R^{b}; alkyl amide of the formula —(CH₂)ₙC(O)NR^{a}R^{b}; thioamide of the formula —C(S)NR^{a}R^{b}; alkyl thioamide of the formula —(CH₂)ₙC(S)NR^{a}R^{b}; aldehyde of the formula —C(O)H; alkyl aldehyde of the formula —(CH₂)ₙC(O)H; thial of the formula —C(S)H; alkyl thial of the formula —(CH₂)ₙC(S)H; ketone of the formula —C(O)R^{a}; alkyl ketone of the formula —(CH₂)ₙC(O) R^{a}; thioketone of the formula —C(S)R^{a}; alkyl thioketone of the formula —(CH₂)nC(S)R^{a}; acetal, of one of the formulae:

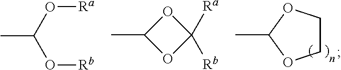

alkyl acetal, of one of the formulae:

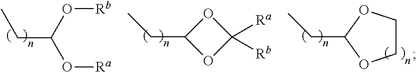

alkyl dithioacetals, of one of the formulae:

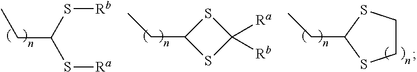

amine of the formula —NR^{a}R^{b}; alkyl amine of the formula —(CH₂)ₙNR^{a}R^{b}; ammonium salt of the formula —N⁺R^{a}R^{b}R^{c} G⁻; alkyl ammonium salt of the formula —(CH₂)ₙN⁺R^{a}R^{b}R^{c} G⁻; amide of the formula —NR^{a}C(O)R^{b}; alkyl amide of the formula —(CH₂)ₙNR^{a}C(O)R^{b}; thioamide of the formula —NR^{a}C(S)R^{b}; alkyl thioamide of one of the formulae:

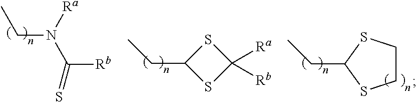

imine of one of the formulae:

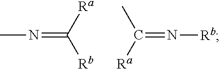

alkyl imine, of one of the formulae:

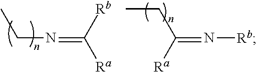

guanidine of the formula

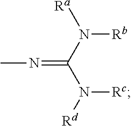

alkyl guanidine of the formula

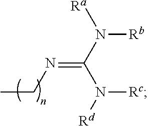

amidine of the formula

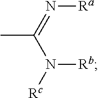

alkyl amidine of the formula

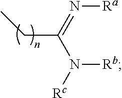

nitrile (cyano) of the formula —CN; alkyl nitrile of the formula —(CH₂)ₙCN; isonitrile of the formula —N⁺C⁻; alkyl isonitrile of the formula —(CH₂)ₙN⁺C⁻; cyanate of the formula —OCN; alkyl cyanate of the formula —(CH₂)ₙOCN; isocyanate of the formula —N═C═O; alkyl isocyanate of the formula —(CH₂)ₙN═C═O; thiocyanate of the formula —SCN; alkyl thiocyanate of the formula —(CH₂)ₙ SCN; isothiocyanate of the formula —N═C═S; alkylisothiocyanate of the formula —(CH₂)ₙN═C═S; azo of the formula —N═NH; alkyl azo of the formula —(CH₂)ₙN═NH; nitro of the formula —NO₂; alkyl nitro of the formula —(CH₂)ₙNO₂; nitrite of the formula —O—N═O; alkyl nitrite of the formula —(CH₂)ₙO—N═O; nitriso of the formula —N═O; alkyl nitriso of the formula —(CH₂)ₙN═O; N-terminal peptide sequences of the formula

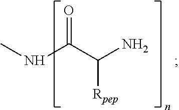

C-terminal peptide sequences of the formula

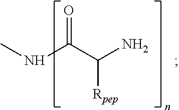

N-terminal peptide alkyl sequence of the formula

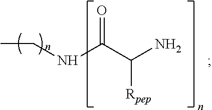

C-terminal peptide alkyl sequence of the formula

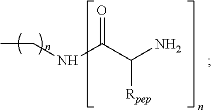

phosphine of the formula —PR^{a}R^{b}; alkyl phosphine of the formula —(CH₂)ₙPR^{a}R^{b}; phosphonium salt of the formula —P⁺R^{a}R^{b}R^{c} G⁻; alkyl phosphonium salt of the formula —(CH₂)ₙP⁺R^{a}R^{b}R^{c} G⁻; phosphine oxide of the formula —P(O)R^{a}R^{b}; alkylphosphine oxide of the formula —(CH₂)ₙP(O)R^{a}R^{b}; phosphites of the formula —O—P(OR^{a})OR^{b}; alkyl phosphites of the formula —(CH₂)ₙO—P(OR^{a})OR^{b}; phosphate of the formula —OP(O)(OR^{a})(OR^{b}); alkyl phosphate of the formula —(CH₂)ₙOP(O)(OR^{a})(OR^{b}); phosphinites of one of the formulae —OPR^{a}R^{b} or —P(R^{a})OR^{b}; alkyl phosphinites of one of the formulae —(CH₂)ₙOPR^{a}R^{b} or −(CH₂)ₙ P(R^{a})OR^{b}; phosphinates of one of the formulae —OP(O)(R^{a})(R^{b}) or —P(O)(R^{a})(OR^{b}); alkyl phosphinates of one of the formulae —(CH₂)ₙOP(O)(R^{a})(R^{b}) or —(CH₂)ₙP(O)(R^{a})(OR^{b}); phosphinites of one of the formulae —OP(OR^{a})(R^{b}) or —P(OR^{a})(OR^{b}); alkyl phosphinites of one of the formulae —(CH₂)ₙOP(OR^{a})(R^{b}) or —(CH₂)ₙP(OR^{a})(OR^{b}); phosphonates of one of the formulae —OP(O)(OR^{a})(R^{b}) or —P(O)(OR^{a})(OR^{b}); alkyl phosphonates of one of the formulae —(CH₂)ₙOP(O)(OR^{a})(R^{b}) or —(CH₂)ₙP(O)(OR^{a})(OR^{b}); sulfate of the formula —S(O)(O)R^{a}; alkyl sulfate of the formula —(CH₂)ₙS(O)(O)R^{a}; sulfone of the formula —S(O)(O)R^{a}; alkyl sulfone of the formula —(CH₂)ₙS(O)(O)R^{a}; sulfoxide of the formula —S(O)R^{a}; alkyl sulfoxide of the formula —(CH₂)ₙS(O)R^{a}; sulfinic acid of the formula —S(O)OR^{a}; alkyl sulfinic acid of the formula —(CH₂)ₙS(O)OR^{a}; sulfimine of one of the formulae —S(R^{a})═N—R^{b} or N═SR^{a}R^{b}; alkyl sulfimine of one of the formulae —(CH₂)ₙS(R^{a})═N—R^{b} or —(CH₂)ₙN═SR^{a}R^{b}; sulfonamide of the formula —S(O)(O)NR^{a}R^{b}; alkyl sulfonamide of the formula —(CH₂)ₙS(O)(O)NR^{a}R^{b}; boronic acid of the formula —B(OH)₂; alklyl boronic acid of the formula —(CH₂)ₙB(OH)₂; boronic ester of the formula —B(OR^{a})(OR^{b}); alkyl boronic ester of the formula —(CH₂)ₙB(OR^{a})(OR^{b}); semicarbazones of the formula

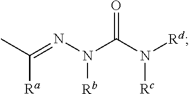

alkyl semicarbazones of the formula

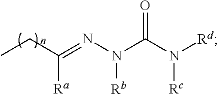

thiosemicarbazones of the formula

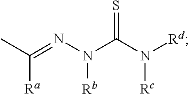

alkyl thiosemicarbazones of the formula

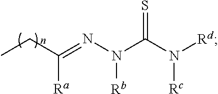

cyanimide of the formula —N(R^{a})CN; alkyl cyanimide of the formula —(CH₂)ₙN(R^{a})CN; hydrazone of the formula —C(R^{a})═N—NH₂; alkyl hydrazone of the formula —(CH₂)ₙC(R^{a})═N—NH₂; oxime of the formula —C(R^{a})═N—OH; alkyl oxime of the formula —(CH₂)ₙC(R^{a})═N—OH; nitraomine of the formula —N(R^{a})NO₂; alkyl nitraomine of the formula —(CH₂)ₙN(R^{a})NO₂; nitronate of the formula

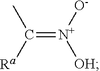

alkyl nitronate of the formula

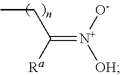

nitrone of the formula

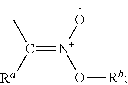

alkyl nitrone of the formula

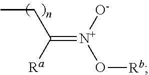

carbonate of the formula —OC(O)OR^{a}; alkyl carbonate of the formula —(CH₂)ₙOC(O)OR^{a}; carbamate of one of the formulae —OC(O)NR^{a}R^{b} or —N(R^{a})C(O)OR^{b}; alkyl carbamate of one of the formulae —(CH₂)ₙOC(O)NR^{a}R^{b} or —(CH₂)ₙN(R^{a})C(O)OR^{b}; dithiocarbamate of one or more of the formulae —SC(S)NR^{a}R^{b} or —N(R^{a})C(S)SR^{b}; alkyl dithiocarbamate of one or more of the formulae —(CH₂)ₙSC(S)NR^{a}R^{b} or —(CH₂)ₙN(R^{a})C(S)SR^{b}; substituted methane of the formula —CR^{r}R^{s}R^{t}; alkyl substituted methane of the formula —(CH₂)ₙCR^{r}R^{s}R^{t}; hydroxyl of the formula —OH; alkylhydroxyl of the formula —(CH₂)ₙOH; ether of the formula —OR^{a}; alkyl ether of the formula —(CH₂)ₙOR^{a}; amide of the formula —NH—C(O)—R^{b}; alkyl amide of the formula —(CH₂)ₙNH—C(O)—R^{b}; ester of the formula —OC(O)R^{b}; alkyl ester of the formula —(CH₂)ₙOC(O)R^{b}; phenyl; alkyl phenyl of the formula —(CH₂)ₙPh; vinyl of the formula —C═C(R^{a})(R^{b}); alkyl vinyl of the formula —(CH₂)ₙC═C(R^{a})(R^{b}); a 5- or 6-membered saturated heterocyclic ring containing 1 to 3 heteroatoms chosen independently from nitrogen, sulfur and oxygen, a 5- or 6-membered saturated heteroaromatic ring containing 1 to 5 heteroatoms chosen independently from nitrogen, sulfur and oxygen, a 5-membered unsaturated heterocyclic ring containing, one or two double bonds and containing 1 to 5 heteroatoms chosen independently from nitrogen, sulphur and oxygen, a 6 membered ring containing 1 to 3 double bonds and containing 1 to 5 heteroatoms chosen independently from nitrogen, sulfur and oxygen dependant on ring size;
wherein R^{a}, R^{b}, R^{c} and R^{d} are independently selected from; H or C₁₋₄ alkyl, Rₚₑₚ is any group resulting in the formation of an amino acid; X is a halogen; and n=1-4; and R^{r}, R^{s} and R^{t} are independently selected from H, C₁₋₄ alkyl or a halogen, with at least one of R^{r}, R^{s} or R^{t} being a halogen; G⁻ is an anion;
and wherein, on any one of W1, W2 or W where none of R₁, R₂, R₃, R₄ or R₅, is selected from the group; alkylphosphines of the formula

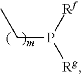

alkyl amines of the formula

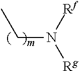

wherein each of R^{f} and R^{g} is independently selected from the group:

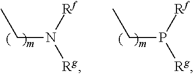

H or C₁-C₈ alkyl; and m is 1-8; none of R₁, R₂, R₃, R₄ or R₅ may be selected from the group;
halogen selected from the group F, Cl, Br, I; alkyl halogen of the formula —(CH₂)ₙ—X; carboxylic acid of the formula —C(O)OH, alkyl carboxylic acid of the formula —(CH₂)ₙC(O)OH; ester of the formula —OC(O)R^{b}; alkyl ester of the formula —(CH₂)ₙOC(O)R^{b}; aldehyde of the formula —C(O)H; alkyl aldehyde of the formula —(CH₂)ₙC(O)H; ketone of the formula —C(O)R^{a}; alkyl ketone of the formula —(CH₂)ₙC(O) R^{a}; sulfate of the formula —S(O)(O)R^{a}; alkyl sulfate of the formula —(CH₂)ₙS(O)(O)R^{a}; nitrile (cyano) of the formula —CN; alkyl nitrile of the formula —(CH₂)ₙCN; nitro of the formula —NO₂; alkyl nitro of the formula —(CH₂)ₙNO₂; amide of the formula —C(O)NR^{a}R^{b}; alkyl amide of the formula —(CH₂)ₙC(O)NR^{a}R^{b}; amide of the formula —NR^{a}C(O)R^{b}; alkyl amide of the formula —(CH₂)ₙNR^{a}C(O)R^{b};
and wherein R^{a}, R^{b}, R^{c} and R^{d} are independently selected from H or C₁₋₄ alkyl; X is a halogen; and n=1-4
or pharmaceutically acceptable salts thereof.

The compounds of the present invention have antimicrobial activity, and thus are useful for treatment or prevention of Gram-negative bacterial infections. Thus, the present invention also provides a method of treating or preventing a Gram-negative bacterial infection in a subject comprising the step of administering to the subject an effective amount of a compound of Formula (I) or pharmaceutically acceptable salts described herein.

The present invention further provides the use of a compound of Formula (I), or pharmaceutically acceptable salts thereof in the manufacture of a medicament for the therapeutic treatment or prevention of bacterial infection or disease in a subject in need thereof, wherein the bacterial infection or disease results from Gram-negative bacteria.

The present invention provides a compound of Formula (I), or pharmaceutically acceptable salts thereof for use in a method of treating or preventing Gram-negative bacterial infections in a subject in need thereof, the method including the step of administering a therapeutically effective amount of a compound of Formula (I), or a therapeutically acceptable salt thereof, to the subject, wherein the bacterial infection or disease results from Gram-negative bacteria.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 provides a general pathway for synthesis of the target compounds through the Heck Cross coupling approach.

FIG. 2 provides structure for a compound according to Formula (I).

FIG. 3 provides structure for a compound according to Formula (I).

FIGS. 4A and 4B provide structures for compounds according to Formula (I).

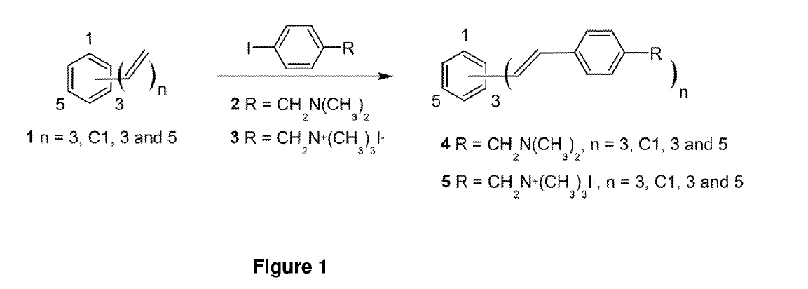

### DESCRIPTION OF INVENTION

### Detailed Description of the Invention

### Compounds

The present invention provides a compound of Formula (I):

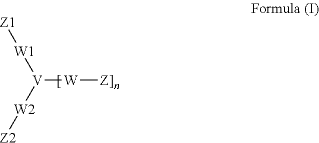

wherein;
V is covalently bonded to W;
W is covalently bonded to Z;
V is a six membered, aromatic ring;
n is 0,1, 2, 3, or 4;
the, or each, W is independently selected from the group;
C₂₋₄ alkyl groups; C₂₋₄ substituted alkyl groups; C₂ E-alkene, with V and Z in the 1 and 2 positions; C₂ Z-alkene, with V and Z in the 1 and 2 positions; C₂-alkyne;
the, or each, Z is independently selected from the group;

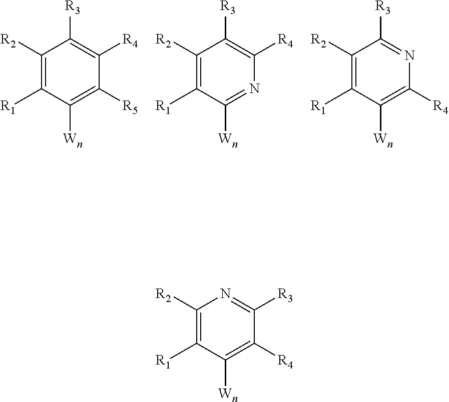

wherein Wₙ is one of W1, W2 or the or each W;
and wherein;
at least one of R₁, R₂, R₃, R₄ or R₅, on at least one of W1, W2 and the or each W is selected from the group;
alkylphosphines of the formula

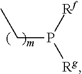

alkyl amines of the formula

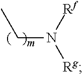

wherein each of R^{f} and R^{g} is independently selected from;

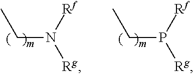

H, C₁-C₈ alkyl; and m is 1-8;
and wherein;
each of the remaining R₁, R₂, R₃, R₄ or R₅ is independently selected from;
halogen selected from the group F, Cl, Br, I; C₁₋₈ alkyl; carboxylic acid of the formula —C(O)OH; alkyl carboxylic acid of the formula —(CH₂)ₙC(O)OH; thiocarboxylic acid of the formula —C(S)OH; Alkyl thiocarboxylic acid of the formula —(CH₂)ₙC(S)OH; ester of the formula —C(O)OR^{a}; alkyl ester of the formula —(CH₂)ₙC(O)OR^{a}; Thioester of the formula —C(S)OR^{a}; alkyl thioester of the formula —(CH₂)ₙC(S)OR^{a}; dithioester of the formula —C(S)SR^{a}; alkyl dithioester of the formula —(CH₂)ₙC(S)SR^{a}; amide of the formula —C(O)NR^{a}R^{b}; alkyl amide of the formula —(CH₂)ₙC(O)NR^{a}R^{b}; thioamide of the formula —C(S)NR^{a}R^{b}; alkyl thioamide of the formula —(CH₂)ₙC(S)NR^{a}R^{b}; aldehyde of the formula —C(O)H; alkyl aldehyde of the formula —(CH₂)ₙC(O)H; thial of the formula —C(S)H; alkyl thial of the formula —(CH₂)ₙC(S)H; ketone of the formula —C(O)R^{a}; alkyl ketone of the formula —(CH₂)ₙC(O) R^{a}; thioketone of the formula —C(S)R^{a}; alkyl thioketone of the formula —(CH₂)nC(S)R^{a}; acetal, of one of the formulae:

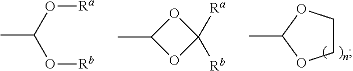

alkyl acetal, of one of the formulae:

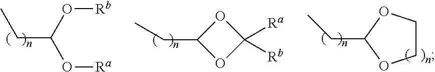

alkyl dithioacetals, of one of the formulae:

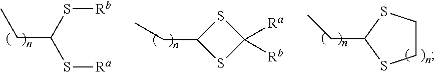

amine of the formula —NR^{a}R^{b}; alkyl amine of the formula —(CH₂)ₙNR^{a}R^{b}; ammonium salt of the formula —N⁺R^{a}R^{b}R^{c} G⁻; alkyl ammonium salt of the formula —(CH₂)ₙN⁺R^{a}R^{b}R^{c} G⁻; amide of the formula —NR^{a}C(O)R^{b}; alkyl amide of the formula —(CH₂)ₙNR^{a}C(O)R^{b}; thioamide of the formula —NR⁸C(S)R^{b}; alkyl thioamide of one of the formulae:

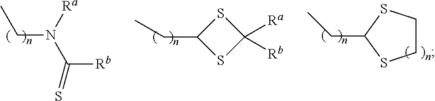

imine of one of the formulae:

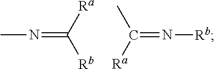

alkyl imine, of one of the formulae:

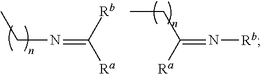

guanidine of the formula

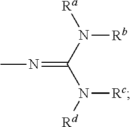

alkyl guanidine of the formula

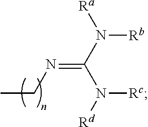

amidine of the formula

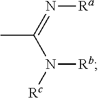

alkyl amidine of the formula

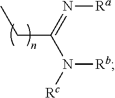

nitrile (cyano) of the formula —CN; alkyl nitrile of the formula —(CH₂)ₙCN; isonitrile of the formula —N⁺C⁻; alkyl isonitrile of the formula —(CH₂)ₙN⁺C⁻; cyanate of the formula —OCN; alkyl cyanate of the formula —(CH₂)ₙOCN; isocyanate of the formula —N═C═O; alkyl isocyanate of the formula —(CH₂)ₙN═C═O; thiocyanate of the formula —SCN; alkyl thiocyanate of the formula —(CH₂)ₙ SCN; isothiocyanate of the formula —N═C═S; alkylisothiocyanate of the formula —(CH₂)ₙN═C═S; azo of the formula —N═NH; alkyl azo of the formula —(CH₂)ₙN═NH; nitro of the formula —NO₂; alkyl nitro of the formula —(CH₂)ₙNO₂; nitrite of the formula —O—N═O; alkyl nitrite of the formula —(CH₂)ₙO—N═O; nitriso of the formula —N═O; alkyl nitriso of the formula —(CH₂)ₙN═O; N-terminal peptide sequences of the formula

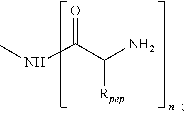

C-terminal peptide sequences of the formula

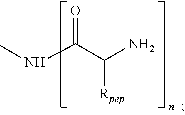

N-terminal peptide alkyl sequence of the formula

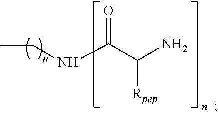

C-terminal peptide alkyl sequence of the formula

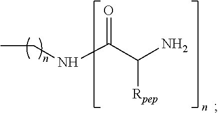

phosphine of the formula —PR^{a}R^{b}; alkyl phosphine of the formula —(CH₂)ₙPR^{a}R^{b}; phosphonium salt of the formula —P⁺R^{a}R^{b}R^{c} G⁻; alkyl phosphonium salt of the formula —(CH₂)ₙP⁺R^{a}R^{b}R^{c} G⁻; phosphine oxide of the formula —P(O)R^{a}R^{b}; alkylphosphine oxide of the formula —(CH₂)ₙP(O)R^{a}R^{b}; phosphites of the formula —O—P(OR^{a})OR^{b}; alkyl phosphites of the formula —(CH₂)ₙO—P(OR^{a})OR^{b}; phosphate of the formula —OP(O)(OR^{a})(OR^{b}); alkyl phosphate of the formula —(CH₂)ₙOP(O)(OR^{a})(OR^{b}); phosphinites of one of the formulae —OPR^{a}R^{b} or —P(R^{a})OR^{b}; alkyl phosphinites of one of the formulae —(CH₂)ₙOPR^{a}R^{b} or —(CH₂)ₙ P(R^{a})OR^{b}; phosphinates of one of the formulae —OP(O)(R^{a})(R^{b}) or —P(O)(R^{a})(OR^{b}); alkyl phosphinates of one of the formulae —(CH₂)ₙOP(O)(R^{a})(R^{b}) or —(CH₂)ₙP(O)(R^{a})(OR^{b}); phosphinites of one of the formulae —OP(OR^{a})(R^{b}) or —P(OR^{a})(OR^{b}); alkyl phosphinites of one of the formulae —(CH₂)ₙOP(OR^{a})(R^{b}) or —(CH₂)ₙP(OR^{a})(OR^{b}); phosphonates of one of the formulae —OP(O)(OR^{a})(R^{b}) or —P(O)(OR^{a})(OR^{b}); alkyl phosphonates of one of the formulae —(CH₂)ₙOP(O)(OR^{a})(R^{b}) or —(CH₂)ₙP(O)(OR^{a})(OR^{b}); sulfate of the formula —S(O)(O)R^{a}; alkyl sulfate of the formula —(CH₂)ₙS(O)(O)R^{a}; sulfone of the formula —S(O)(O)R^{a}; alkyl sulfone of the formula —(CH₂)ₙS(O)(O)R^{a}; sulfoxide of the formula —S(O)R^{a}; alkyl sulfoxide of the formula —(CH₂)ₙS(O)R^{a}; sulfinic acid of the formula —S(O)OR^{a}; alkyl sulfinic acid of the formula —(CH₂)ₙS(O)OR^{a}; sulfimine of one of the formulae —S(R^{a})═N—R^{b} or —N═SR^{a}R^{b}; alkyl sulfimine of one of the formulae —(CH₂)ₙS(R^{a})═N—R^{b} or —(CH₂)ₙN═SR^{a}R^{b}; sulfonamide of the formula —S(O)(O)NR^{a}R¹³; alkyl sulfonamide of the formula —(CH₂)ₙS(O)(O)NR^{a}R^{b}; boronic acid of the formula —B(OH)₂; alklyl boronic acid of the formula —(CH₂)ₙB(OH)₂; boronic ester of the formula —B(OR^{a})(OR^{b}); alkyl boronic ester of the formula —(CH₂)ₙB(OR^{a})(OR^{b}); semicarbazones of the formula

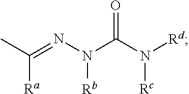

alkyl semicarbazones of the formula

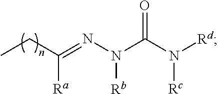

thiosemicarbazones of the formula

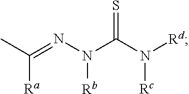

alkyl thiosemicarbazones of the formula

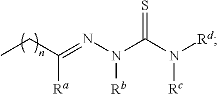

cyanimide of the formula —N(R^{a})CN; alkyl cyanimide of the formula —(CH₂)ₙN(R^{a})CN; hydrazone of the formula —C(R^{a})═N—NH₂; alkyl hydrazone of the formula —(CH₂)ₙC(R^{a})═N—NH₂; oxime of the formula —C(R^{a})═N—OH; alkyl oxime of the formula —(CH₂)ₙC(R^{a})═N—OH; nitraomine of the formula —N(R^{a})NO₂; alkyl nitraomine of the formula —(CH₂)ₙN(R^{a})NO₂; nitronate of the formula

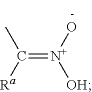

alkyl nitronate of the formula

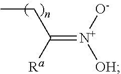

nitrone of the formula

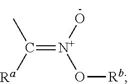

alkyl nitrone of the formula

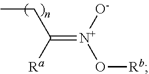

carbonate of the formula —OC(O)OR^{a}; alkyl carbonate of the formula —(CH₂)ₙOC(O)OR^{a}; carbamate of one of the formulae —OC(O)NR^{a}R^{b} or —N(R^{a})C(O)OR^{b}; alkyl carbamate of one of the formulae —(CH₂)ₙOC(O)NR^{a}R^{b} or —(CH₂)ₙN(R^{a})C(O)OR^{b}; dithiocarbamate of one or more of the formulae —SC(S)NR^{a}R^{b} or —N(R^{a})C(S)SR^{b};alkyl dithiocarbamate of one or more of the formulae —(CH₂)ₙSC(S)NR^{a}R^{b} or —(CH₂)ₙN(R^{a})C(S)SR^{b}; substituted methane of the formula —CR^{r}R^{s}R^{t}; alkyl substituted methane of the formula —(CH₂)ₙCR^{r}R^{s}R^{t}; hydroxyl of the formula —OH; alkylhydroxyl of the formula —(CH₂)ₙOH; ether of the formula —OR^{a}; alkyl ether of the formula —(CH₂)ₙOR^{a}; amide of the formula —NH—C(O)—R^{b}; alkyl amide of the formula —(CH₂)ₙNH—C(O)—R^{b}; ester of the formula —OC(O)R^{b}; alkyl ester of the formula —(CH₂)ₙOC(O)R^{b}; phenyl; alkyl phenyl of the formula —(CH₂)ₙPh; vinyl of the formula —C═C(R^{a})(R^{b}); alkyl vinyl of the formula —(CH₂)ₙC═C(R^{a})(R^{b}); a 5- or 6-membered saturated heterocyclic ring containing 1 to 3 heteroatoms chosen independently from nitrogen, sulfur and oxygen, a 5- or 6-membered saturated heteroaromatic ring containing 1 to 5 heteroatoms chosen independently from nitrogen, sulfur and oxygen, a 5-membered unsaturated heterocyclic ring containing, one or two double bonds and containing 1 to 5 heteroatoms chosen independently from nitrogen, sulphur and oxygen, a 6 membered ring containing 1 to 3 double bonds and containing 1 to 5 heteroatoms chosen independently from nitrogen, sulfur and oxygen dependant on ring size;
wherein R^{a}, R^{b}, R^{c} and R^{d} are independently selected from; H or C₁₋₄ alkyl, Rₚₑₚ is any group resulting in the formation of an amino acid; X is a halogen; and n=1-4; and R^{r}, R^{s} and R^{t} are independently selected from H, C₁₋₄ alkyl or a halogen, with at least one of R^{r}, R^{s} or R^{t} being a halogen; G⁻ is an anion;
and wherein, on any one of W1, W2 or W where none of R₁, R₂, R₃, R₄ or R₅, is selected from the group; alkylphosphines of the formula

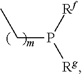

alkyl amines of the formula

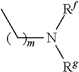

wherein each of R^{f} and R^{g} is independently selected from the group:

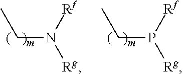

H or C₁-C₈ alkyl; and m is 1-8; none of R₁, R₂, R₃, R₄ or R₅ may be selected from the group;
halogen selected from the group F, Cl, Br, I; alkyl halogen of the formula —(CH₂)ₙ—X; carboxylic acid of the formula —C(O)OH, alkyl carboxylic acid of the formula —(CH₂)ₙC(O)OH; ester of the formula —OC(O)R^{b}; alkyl ester of the formula —(CH₂)ₙOC(O)R^{b}; aldehyde of the formula —C(O)H; alkyl aldehyde of the formula —(CH₂)ₙC(O)H; ketone of the formula —C(O)R^{a}; alkyl ketone of the formula —(CH₂)ₙC(O) R^{a}; sulfate of the formula —S(O)(O)R^{a}; alkyl sulfate of the formula —(CH₂)ₙS(O)(O)R^{a}; nitrile (cyano) of the formula —CN; alkyl nitrile of the formula —(CH₂)ₙCN; nitro of the formula —NO₂; alkyl nitro of the formula —(CH₂)ₙNO₂; amide of the formula —C(O)NR^{a}R^{b}; alkyl amide of the formula —(CH₂)ₙC(O)NR^{a}R^{b}; amide of the formula —NR^{a}C(O)R^{b}; alkyl amide of the formula —(CH₂)ₙNR^{a}C(O)R^{b};
and wherein R^{a}, R^{b}, R^{c} and R^{d} are independently selected from H or C₁₋₄ alkyl; X is a halogen; and n=1-4;
or pharmaceutically acceptable salts thereof.

Without wishing to be bound by theory, it is believed that the activity of the compounds of Formula (I) is substantially dependent on the localisation of the lone pair of electrons of the alkyl amine and alkyl phosphine R groups on the peripheral W rings. Conjugated bond arrangements inherent with phosphines and amines are avoided by the inclusion of the alkyl moiety. While it is not believed that every peripheral W ring need be substituted with an alkyl amine or alkyl phosphine R group, the inventors have observed that certain functional groups, in particular electron withdrawing groups, on unsubstituted rings negate the activity of the compounds.

Further, and without wishing to be bound by theory, the localisation of the lone pair of electrons may be further enhanced by appropriate positioning of electron withdrawing- or electron donating-groups relative to the alkyl amine or alkyl phosphine group, which it is believed will further enhance the activity of compounds of the invention.

Rₚₑₚ preferably has a peptide comprised of one or more α-amino acids, independently selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine.

Pharmaceutically acceptable salts for the purposes of the present invention include non-toxic cation and anion salts. Examples include, but are not limited to sodium, potassium, aluminium, calcium, lithium, magnesium, zinc and from bases such as ammonium, ethylenediamine, N-methyl-glutamine, lysine, arginine, ornithine, choline, N, N′-dibenzylethlenediamine, diethylamine, piperazine, tris(hydroxymethyl)aminomethane, tetramethylammonium, acetate, lactobionate, benzenesulfonate, laurate, benzoate, malate, bicarbonate, maleate, bisulfate, mandelate, bitratrate, meyate, borate, methylbromide, bromide, methylnitrate, calcium edetate, methylsulfate, camsylate, mucate, carbonate, napsylate, chloride, nitrate, clavulanate, N-methylglucamine, citrate, hydrochloride, oleate, edetate, oxalate, edisylate, pamoate (embonate), estolate, palmitate, esylate, pantothenate, fumarate, phosphate, diphosphate, glucepate, plygalacturonate, gluconate, salicylate, glutamate, stearate, glycollylarsanilate, sulfate, hexylresorcinate, subacetate, hydrabamine, succinate, hydrobromide, tannate, tartrate, hydroxynapthoate, teoclate, iodide, tosylate, isothionate, triethiodide, lactate, panoate and valerate.

The compounds of the present invention may be generated using synthesis methods such as those disclosed in PCT/AU2010/001709 and PCT/AU2016/095003 (based on provisional filing AU2015903284).

### Antibiotic Use

The compounds of the present invention have antimicrobial activity, and thus are useful for treatment or prevention of Gram-negative bacterial infections. Thus, the present invention also provides a method of treating or preventing a Gram-negative bacterial infection in a subject comprising the step of administering to the subject an effective amount of a compound of Formula (I) or pharmaceutically acceptable salts.

Examples of Gram-negative bacteria which the compounds of the present invention may preferably be used against include: 86983 Acinetobacter baumannii, Aeromonas hydrophila, Arcobacter species, Bacteriodes species, Brachyspira aalborgi, Brachyspira pilosicoli, Cardiobacterium hominis, Citrobacter freundii, Coxiella bumetii, Enterobacter cloacae, Escherichia coli, Escherichia fergusonii, Fusobacterium necrophorum, Fusobacterium nucleatum, Fusobacterium polymorphum, Haemophilus felis, Haemophilus haemolyticus, Haemophilus influenza, Haemophilus pittmaniae, Helicobacter pylori, Kingella kingae, Klebsiella edwardsii, Klebsiella pneumoniae, Legionella species, Moraxella catarrhalis, Moraxella lacunata, Morganella morganii, Neisseria gonorrhoeae, Neisseria meningitides, Proteus mirabilis, Proteus penneri, Pseudomonas aeruginosa, Pseudomonas luteola, Rickettsia rickettsia, Salmonella species, Serratia marcescens, Shigella species, Stenotrophomonas maltophilia, Vibrio cholerae (non-toxigenic), Yersinia enterocolitica and Yersinia pestis.

Antibiotics of Formula (I) belong to a new class of styrylbenzene-based derivative antibiotics. This generation of antibiotics has shown activity against the Mechanosensitive Ion Channel of Large Conductance (MscL), a novel and highly sought after bacterial target. MscL is a highly conserved transmembrane protein found in all bacteria (including Gram-negative bacteria) but not in the human genome, making it an ideal drug target. The channel is responsible for saving bacterial cells from lysis in a high osmotic environment. It responds to a high turgor pressure by opening up and allowing bacteria to release osmolytes thereby reducing the pressure within. Styrylbenzene-based antibiotics lower the threshold at which these channels open and elongate their opening times, causing the loss of important osmolytes and other biomolecules and thereby weakening the bacteria.

The present invention provides a method of treating or preventing a Gram-negative bacterial infection in a subject comprising the step of administering to the subject an effective amount of a compound of Formula (I) wherein none of R₁ to R₅ of Formula (I) are: the ammonium salt of the formula

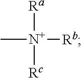

the alkyl ammonium salt of the formula

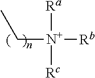

the phosphonium salt of the formula

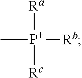

or the alkyl phosponium salt of the formula

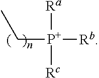

It is believed that these compounds may be toxic and may not be suitable for use as antibiotic therapeutics. However, it is possible to use these compounds for non-in vivo uses, such as spraying on surfaces to remove Gram-negative bacterial contamination.

### Manufacture of Medicament

The present invention further provides the use of a compound of Formula (I), or pharmaceutically acceptable salts thereof in the manufacture of a medicament for the therapeutic treatment or prevention of bacterial infection or disease in a subject in need thereof, wherein the bacterial infection or disease results from Gram-negative bacteria.

The present invention also provides a compound of Formula (I), or pharmaceutically acceptable salts thereof for use in a method of treating or preventing Gram-negative bacterial infections in a subject in need thereof, the method including the step of administering a therapeutically effective amount of a compound of Formula (I), or a therapeutically acceptable salt thereof, to the subject, wherein the bacterial infection or disease results from Gram-negative bacteria.

### Formulations

The compounds of the present invention may be made into formulations for administration.

Thus, the present invention also provides a formulation comprising a therapeutically-effective amount of a compound of Formula (I) and pharmaceutically acceptable salts thereof, and a pharmaceutically acceptable carrier or diluent. The formulation will preferably treat or prevent an infection with a Gram-negative bacteria.

The precise formulation of the present invention will vary according to a wide range of commercial and scientific criteria. Methods for the preparation of pharmaceutical formulations comprising one or more active ingredients are generally known in the art. Such formulations will generally be formulated for the mode of delivery that is to be used and will usually include one or more pharmaceutically acceptable carriers.

Generally, examples of suitable carriers, excipient and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, talc magnesium stearate and mineral oil or combinations thereof. The formulations can additionally include lubricating agents, pH buffering agents, wetting agents, emulsifying and suspending agents, preserving agents, sweetening agents or flavouring agents.

### (a) Topicals

The pharmaceutical formulation may be adapted for topical application. In this regard, various topical delivery systems may be appropriate for administering the formulations of the present invention depending up on the preferred treatment regimen. Topical formulations may be produced by dissolving or combining the compound of the present invention in an aqueous or nonaqueous carrier. In general, any liquid, cream, or gel or similar substance that does not appreciably react with the compound or any other of the active ingredients that may be introduced into the formulation and which is non-irritating is suitable. Appropriate non-sprayable viscous, semi-solid or solid forms can also be employed that include a carrier compatible with topical application and have dynamic viscosity preferably greater than water.

Suitable formulations are well known to those skilled in the art and include, but are not limited to, solutions, suspensions, emulsions, creams, gels, ointments, powders, liniments, salves, aerosols, transdermal patches, etc, which are, if desired, sterilised or mixed with auxiliary agents, e.g. preservatives, stabilisers, emulsifiers, wetting agents, fragrances, colouring agents, odour controllers, thickeners such as natural gums, etc. Particularly preferred topical formulations include ointments, creams or gels.

Ointments generally are prepared using either (1) an oleaginous base, i.e., one consisting of fixed oils or hydrocarbons, such as white petroleum or mineral oil, or (2) an absorbent base, i.e., one consisting of an anhydrous substance or substances which can absorb water, for example anhydrous lanolin. Customarily, following formation of the base, whether oleaginous or absorbent, the active ingredient is added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulfite; hydrophilic colloids, such as acacia colloidal clays, veegum and the like. Upon formation of the emulsion, the compound can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the compound is added to the formulation at the desired concentration at a point preceding addition of the gelling agent.

The amount of compound incorporated into a topical formulation is not critical; the concentration should be within a range sufficient to permit ready application of the formulation such that an effective amount of the compound is delivered.

### (b) Oral Formulations

The pharmaceutical formulation may be adapted for oral delivery. In this regard, the compound can be administered as an oral preparation adapted in such a manner that facilitates delivery of a therapeutically effective concentration of the compound.

The effective dosages of the compound, when administered orally, must take into consideration the diluent, preferably water. The formulation preferably contains 0.05% to about 100% by weight active ingredient and more preferably about 10% to about 80% by weight. When the formulations are ingested, desirably they are taken on an empty stomach.

Contemplated for use herein are oral solid dosage forms including tablets, capsules, pills, troches or lozenges, cachets or pellets. Also, liposomal or proteinoid encapsulation may be used to formulate the present formulations. Liposomal encapsulation may be used and the liposomes may be derivatised with various polymers. In general, the formulation will include the compound and inert ingredients that allow for protection against the stomach environment and release of the biologically active material in the intestine.

The location of release may be the stomach, the small intestine (the duodenum, the jejunem, or the ileum), or the large intestine. One skilled in the art has available formulations that will not dissolve in the stomach, yet will release the material in the duodenum or elsewhere in the intestine. Preferably, the release will avoid the deleterious effects of the stomach environment, either by protection of the formulation or by release of the compound beyond the stomach environment, such as in the intestine.

To ensure full gastric resistance, a coating impermeable to at least pH 5.0 may be used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S and Shellac. These coatings may be used as mixed films.

A coating or mixture of coatings that are not intended for protection against the stomach can also be used on tablets. This can include sugar coatings, or coatings that make the tablet easier to swallow. Capsules may consist of a hard shell (such as gelatine) for delivery of dry therapeutic i.e. powder; for liquid forms, a soft gelatine shell may be used. The shell material of cachets could be thick starch or other edible paper. For pills, lozenges, moulded tablets or tablet triturates, moist massing techniques can be used.

One may dilute or increase the volume of the formulation with an inert material. These diluents could include carbohydrates, especially mannitol, alpha-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the compound into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite may all be used. Another form of the disintegrants is insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the formulation together to form a hard tablet and include materials from natural products such as acacia, tragacanth, starch and gelatine. Others include methylcellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the compound.

An antifrictional agent may be included in the formulation to prevent sticking during the formulation process. Lubricants may be used as a layer between the compound and the die wall and these can include but are not limited to: stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights and Carbowax 4000 and 6000.

Glidants that might improve the flow properties of the formulation during formulation and to aid rearrangement during compression might be added. The glidants may include starch, talc, pyrogenic silica and hydrated silicoaluminate.

To aid dissolution of the compound, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation either alone or as a mixture in different ratios.

Controlled release formulations may be desirable. The compounds can be incorporated into an inert matrix that permits release by either diffusion or leaching mechanisms i.e., gums. Slowly degenerating matrices may also be incorporated into the formulation. Another form of a controlled release formulation is by a method based on the Oros therapeutic system (Alza Corp.), i.e. the formulation is enclosed in a semipermeable membrane which allows water to enter and push the formulation out through a single small opening due to osmotic effects. Some enteric coatings also have a delayed release effect.

A mix of materials might be used to provide the optimum film coating. Film coating may be carried out in a pan coater or in a fluidised bed or by compression coating.

The compound can be included in the formulation as fine multiparticulates in the form of granules or pellets of particle size about 1 mm. The formulation of the material for capsule administration could also be as a powder, lightly compressed plugs or even as tablets. The compound could be prepared by compression.

### (c) Injectable Formulations

The compound can also be formulated for parenteral delivery. Pharmaceutical forms suitable for injectable use include: sterile aqueous solutions (where water-soluble) or dispersions and sterile powders for the extemporaneous preparation of sterile injectable solutions or dispersion. Alternatively, the compounds of the invention may be encapsulated in liposomes and delivered in injectable solutions to assist their transport across cell membrane. The solution may be a solvent or dispersion medium containing, for example, water, ethanol, polyol (for example, glycerol, propylene glycol and liquid polyethylene glycol and the like), suitable mixtures thereof and vegetable oils. Proper fluidity may be maintained, for example, by the use of a coating such as lecithin, by the maintenance of the required particle size in the case of dispersion and by the use of surfactants. Prolonged absorption of the injectable formulations can be brought about by the use in the formulations of agents delaying absorption, for example, aluminium monostearate and gelatine.

Sterile injectable solutions may be prepared by incorporating the active compounds in the required amount in an appropriate solvent with various of the other ingredients enumerated above, as required, followed by filtered sterilisation. Generally, dispersions are prepared by incorporating the compound into a sterile vehicle that contains the basic dispersion medium and the other ingredients. In the case of sterile powders for the preparation of sterile injectable solutions, the preferred methods of preparation are vacuum drying and freeze-drying techniques that yield a powder of the compound plus any additional desired ingredient from previously sterile-filtered solution thereof.

Thus, the present invention also provides an injectable, stable, sterile formulation comprising a compound of Formula (I), or a salt thereof, in a unit dosage form in a sealed container. The compound or salt may be provided in lyophilised form capable of being reconstituted with a suitable pharmaceutically acceptable carrier to form a liquid formulation suitable for injection thereof into a subject. The unit dosage form typically comprises from about 10 mg to about 10 grams of the compound or salt thereof. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

### (d) Aerosols

Pharmaceutical formulations are also provided which are suitable for administration as an aerosol, by inhalation. These formulations comprise a solution or suspension of the desired compound or a salt thereof or a plurality of solid particles of the compound or salt. The desired formulation may be placed in a small chamber and nebulized. Nebulization may be accomplished by compressed air or by ultrasonic energy to form a plurality of liquid droplets or solid particles comprising the compounds or salts.

The solid particles can be obtained by processing solid compound or a salt thereof, in any appropriate manner known in the art, such as by micronization. Commercial nebulizers are also available to provide liquid droplets of any desired size.

The liquid droplets or solid particles should have a particle size in the range of about 0.5 to about 5 microns, preferably from about 1 to about 2 microns. Most preferably, the size of the solid particles or droplets will be from about 1 to about 2 microns. Such particles or droplets may be dispensed by commercially available nebulisers or by other means known to the skilled person.

When the pharmaceutical formulation suitable for administration as an aerosol is in the form of a liquid, the formulation will comprise a water-soluble form of the compound or a salt thereof, in a carrier that comprises water. A surfactant may be present which lowers the surface tension of the formulation sufficiently to result in the formation of droplets within the desired size range when subjected to nebulization.

### (e) Mucosal Delivery

Pharmaceutical formulations are also provided which are suitable for administration via the oral (e.g. buccal, sublingual), rectal, vaginal, nasal or other mucosal surface.

Such formulations may be in the form of freeze-dried wafers, flexible polymer films, fast disintegrating tablets, sprayable solutions etc. Such delivery preferably bypasses the first pass effect of oral delivery via the gastric system.

### (f) Other Agents

In addition, the pharmaceutical formulations discussed above may also include other agents. For example, preservatives, co-solvents, surfactants, oils, humectants, emollients, chelating agents, dyestuffs, stabilizers or antioxidants may be employed. Water soluble preservatives that may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. A surfactant may be Tween 80. Other suitable additives include lubricants and slip agents, such as, for example, magnesium stearate, stearic acid, talc and bentonites; substances which promote disintegration, such as starch or crosslinked polyvinylpyrrolidone; binders, such as, for example, starch, gelatin or linear polyvinylpyrrolidone; and dry binders, such as microcrystalline cellulose.

Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylated hydroxyanisole, butylated hydroxytoluene, etc.

The indications, effective doses, formulations, contraindications, vendors etc, of the compounds in the formulations are available or are known to one skilled in the art. These agents may be present in individual amounts of from about 0.001% to about 5% by weight and preferably about 0.01% to about 2%.

Electrolytes such as, but not limited to, sodium chloride and potassium chloride may also be included in the formulation.

Further, the formulations may contain microbial preservatives. Useful microbial preservatives include methylparaben, propylparaben, and benzyl alcohol. The microbial preservative is typically employed when the formulation is placed in a vial designed for multidose use.

Excipients which may be used are all the physiologically acceptable solid inert substances, either inorganic or organic in nature. Inorganic substances are, for example, sodium chloride; carbonates, such as calcium carbonate, bicarbonates; aluminium oxides; silicic acids; aluminas; precipitated or colloidal silicon dioxide; and phosphates. Organic substances are, for example, sugars; cellulose; foodstuffs and feedstuffs, such as milk powder, animal flours, cereal flours and shredded cereals and starches.

Finally, it will be appreciated that the formulations of the present invention may comprise a plurality of compounds as described herein.

### General

Those skilled in the art will appreciate that the invention described herein is susceptible to variations and modifications other than those specifically described. It is to be understood that the invention includes all such variations and modifications. The invention also includes all of the steps, features, formulations and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, formulations and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

As used herein the term “derived” and “derived from” shall be taken to indicate that a specific integer may be obtained from a particular source albeit not necessarily directly from that source.

As used herein, the singular forms “a,” “an” and “the” include plural references unless the context clearly dictates otherwise.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other than in the operating example, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value; however, inherently contains certain errors necessarily resulting from the standard deviation found in their respective testing measurements

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

### Example 1

### MIC by Broth Microdilution

The antibacterial potency of the test article(s) was measured using the in vitro broth microdilution assay under assay conditions described by the Clinical and Laboratory Standards Institute. In this assay, the Minimum Inhibitory Concentration (MIC) is defined as the lowest concentration of an agent that completely inhibits visible growth in vitro of the microorganism. The test substance was dissolved in 100% DMSO, suspended completely by sonication or vortexing, diluted by 2-fold serial titrations in the same vehicle, for a total of 11 test concentrations. A 4 μL aliquot of each dilution was added to 196 μL of broth medium seeded with the organism suspension in wells of a 96 well plate (bacterial count: 2−8×10⁵ colony forming units/mL final). The final volume was 200 μL in each well and the final DMSO concentration was 2 percent. Following incubation for 1 day at 36° C., the test plates were visually examined and wells were scored for growth or complete growth inhibition to define the minimum inhibitory concentration. Each test substance was evaluated in duplicate and the results were reported as the duplicate test values. Vehicle-control and an active reference agent were used as blank and positive controls, respectively. Microorganisms tested were Acinetobacter baumannii (ATCC 17978), Enterococcus faecalis (ATCC 29212), Enterococcus faecium VanA (ATCC 700221), Escherichia coli (ATCC 25922), Escherichia coli efflux defective DEL-tolC (JW5503), Heamophilus influenzae (ATCC 49247), Klebsiella pneumoniae (ATCC 43816), Pseudomonas aeruginosa (ATCC 27853), Pseudomonas aeruginosa MDR (NTUH-974), Staphylococcus aureus (ATCC 29213), Staphylococcus aureus MRSA (ATCC 33591) and Streptococcous pneumoniae (ATCC 46919). H. influenzae was grown in Haemophilus Test Medium and S. pneumoniae was grown in cation adjusted Muller-Hinton Broth II with 5% lysed horse blood. All other microorganisms were grown in cation adjusted Muller-Hinton Broth II.

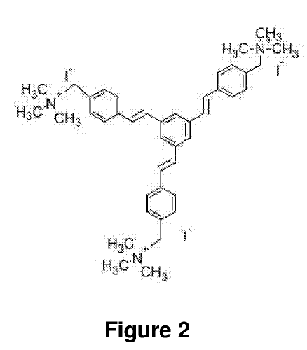

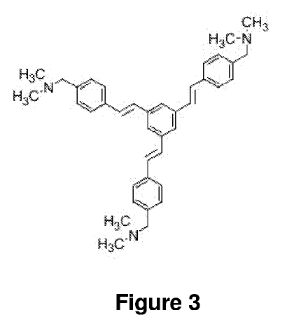

Compound 1 in the below results is shown in FIG. 2 and Compound 2 is shown in FIG. 3. Both compounds are active against Gram-negative bacteria. Without wishing to be bound by theory, this is because Compound 1 has a localized positive charge and Compound 2 has a lone pair of electrons on the nitrogen that cannot be readily delocalized to the ring, making the nitrogen basic.

TABLE 1
Activity of Compounds 1 and 2 against bacteria

| | MIC | |
|---|---|---|
| Species | 1 | 2 |
| Acinetobacter baumannii (ATCC 17978) | 128 | 16 |
| Enterococcus faecalis (ATCC29212) | 128 | 4 |
| Enterococcus faecium, VanA (ATCC 700221) | 128 | 2 |
| Escherichia coli (ATCC 25922) | 64 | 8 |
| Escherichia coli, efflux defective DEL-tolC (JW5503) | 32 | 4 |
| Klebsiella pneumoniae (ATCC 43816) | 128 | 16 |
| Pseudomonas aeruginosa (ATCC 27853) | >256 | 64 |
| Pseudomonas aeruginosa, MDR (NTUH-974) | >256 | 32 |
| Staphylococcus aureus (ATCC 29213) | 32 | 4 |
| Staphylococcus aureus, MRSA (ATCC 33591) MIC | 16 | 4 |
| Streptococcus pneumoniae (ATCC 49619) MIC | >256 | 32 |

### Example 2

### Testing of Compounds

The antibacterial potency of the test article(s) was measured using the in vitro broth microdilution assay under assay conditions described by the Clinical and Laboratory Standards Institute. In this assay, the Minimum Inhibitory Concentration (MIC) is defined as the lowest concentration of an agent that completely inhibits visible growth in vitro of the microorganism. The test substance was dissolved in 100% DMSO (unless stated below), suspended completely by sonication or vortexing, diluted by 2-fold serial titrations in the same vehicle, for a total of 11 test concentrations. A 4 μL aliquot of each dilution was added to 196 μL of broth medium seeded with the organism suspension in wells of a 96 well plate (bacterial count: 2−8×105 colony forming units/mL final). The final volume was 200 μL in each well and the final DMSO concentration was 2 percent. The medium, the incubation time and temperature are listed in the Table of Test Methods. Following incubation, the test plates were visually examined and wells were scored for growth or complete growth inhibition to define the minimum inhibitory concentration. Each test substance was evaluated in duplicate and the results below are the duplicate test values. Vehicle-control and an active reference agent were used as blank and positive controls, respectively.

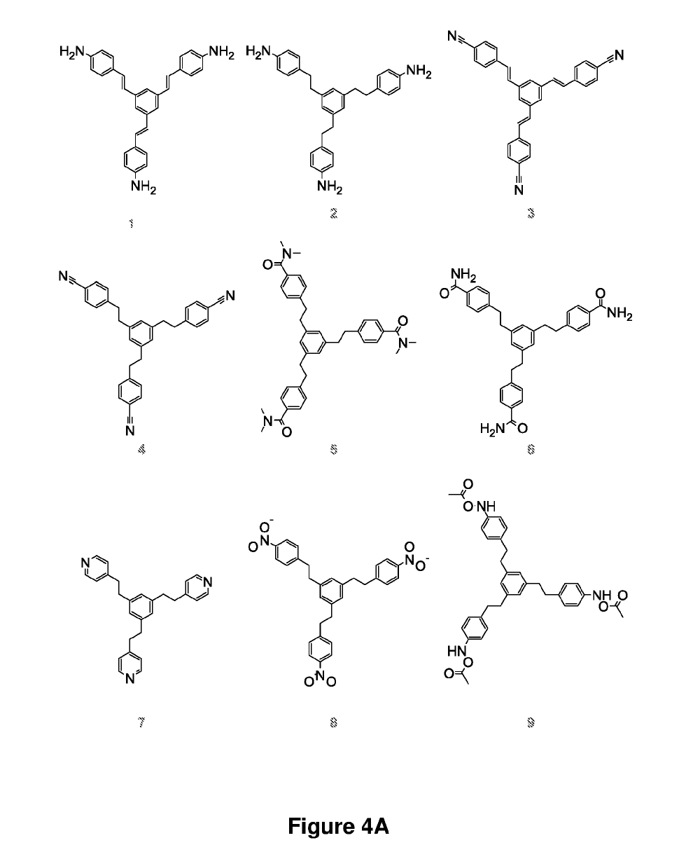

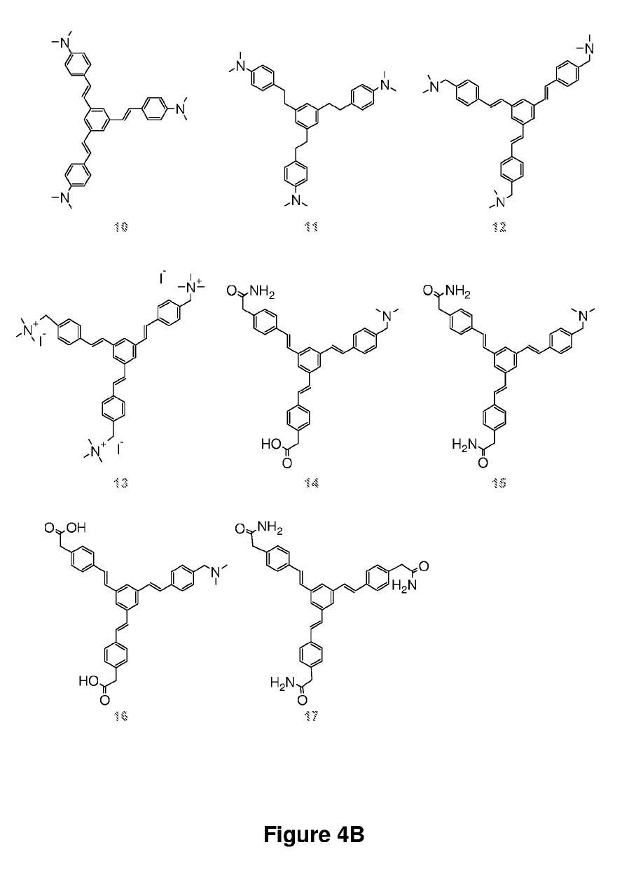

The structures of the compounds tested are provided in FIG. 4.

TABLE 2
Activity of Compounds against bacteria

| | MIC (μg/mL) | | | | | | |
|---|---|---|---|---|---|---|---|
| | 2 | | 4 | 5 | 6 | 8 | 9 |
| Species | (TSB014) | 3 | (TSB064) | (TSB032) | (TSB030) | (TSB036) | (TSB016) |
| Acinetobacter | >32.0 | >128 | >32.0 | >32.0 | >32.0 | >32.0 | >32.0 |
| baumannii | | | | | | | |
| (ATCC 17978) | | | | | | | |
| Escherichia | >32.0 | >128 | >32.0 | >32.0 | >32.0 | >32.0 | >32.0 |
| coli (ATCC | | | | | | | |
| 25922) | | | | | | | |
| Escherichia | >32.0 | >128 | >32.0 | >32.0 | >32.0 | >32.0 | >32.0 |
| coli, efflux | | | | | | | |
| defective DEL- | | | | | | | |
| tolC (JW5503) | | | | | | | |
| MIC | | | | | | | |
| Haemophilus | >32.0 | >128 | >32.0 | >32.0 | >32.0 | >32.0 | >32.0 |
| influenzae | | | | | | | |
| (ATCC 49247) | | | | | | | |
| MIC | | | | | | | |
| Klebsiella | >32.0 | >128 | >32.0 | >32.0 | >32.0 | >32.0 | >32.0 |
| pneumoniae | | | | | | | |
| (ATCC 43816) | | | | | | | |
| Pseudomonas | >32.0 | >128 | >32.0 | >32.0 | >32.0 | >32.0 | >32.0 |
| aeruginosa | | | | | | | |
| (ATCC 27853) | | | | | | | |
| MIC | | | | | | | |
| Pseudomonas | >32.0 | >128 | >32.0 | >32.0 | >32.0 | >32.0 | >32.0 |
| aeruginosa, | | | | | | | |
| MDR (NTUH- | | | | | | | |
| 974) | | | | | | | |

Activity of Compounds against bacteria

| | MIC (μg/mL) | | | | | | | |
|---|---|---|---|---|---|---|---|---|
| | | 11 | | | | | | |
| | 10 | (TSB010) | 12 | 13 | 14 | 15 | 16 | 17 |
| Acinetobacter | >128 | >32.0 | 16 | 128 | >128 | >128 | >128 | >128 |
| baumannii | | | | | | | | |
| (ATCC 17978) | | | | | | | | |
| Escherichia | >128 | >32.0 | 8 | 64 | >128 | >128 | >128 | >128 |
| coli (ATCC | | | | | | | | |
| 25922) | | | | | | | | |
| Escherichia | >128 | >32.0 | 4 | 32 | >128 | >128 | >128 | >128 |
| coli, efflux | | | | | | | | |
| defective DEL- | | | | | | | | |
| tolC (JW5503) | | | | | | | | |
| MIC | | | | | | | | |
| Haemophilus | >128 | >32.0 | >128 | >128 | >128 | >128 | >128 | >128 |
| influenzae | | | | | | | | |
| (ATCC 49247) | | | | | | | | |
| MIC | | | | | | | | |
| Klebsiella | >128 | >32.0 | 16 | 128 | >128 | >128 | >128 | >128 |
| pneumoniae | | | | | | | | |
| (ATCC 43816) | | | | | | | | |
| Pseudomonas | >128 | >32.0 | 64 | >128 | >128 | >128 | >128 | >128 |
| aeruginosa | | | | | | | | |
| (ATCC 27853) | | | | | | | | |
| MIC | | | | | | | | |
| Pseudomonas | >128 | >32.0 | 32 | >128 | >128 | >128 | >128 | >128 |
| aeruginosa, | | | | | | | | |
| MDR (NTUH- | | | | | | | | |
| 974) | | | | | | | | |

As can be seen, the above N containing compounds were ineffective, except for compounds 12 and 13, which correspond to Compounds 1 and 2 discussed in Example 1. Even Compounds 14, 15 and 16, which have one alkyl amine, were ineffective.

### Example 3

1. A compound having a Formula (I): 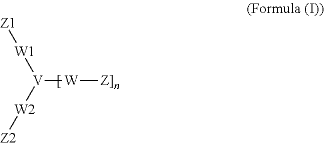
wherein;
V is covalently bonded to W;
W is covalently bonded to Z;
V is a six membered, aromatic ring;
n is 0,1, 2, 3, or 4;
the, or each, W is independently selected from the group;
C₂₋₄ alkyl groups; C₂₋₄ substituted alkyl groups; C₂ E-alkene, with V and Z in the 1 and 2 positions; C₂ Z-alkene, with V and Z in the 1 and 2 positions; C₂-alkyne;
the, or each, Z is independently selected from the group; 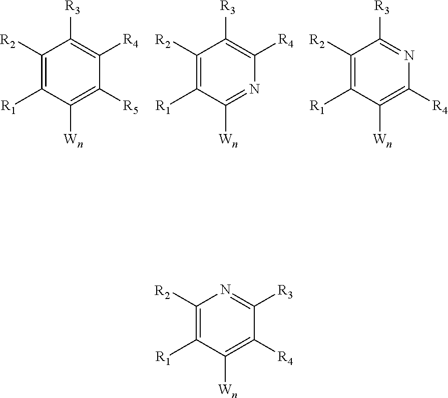
wherein Wₙ is one of W1, W2 or the or each W;
and wherein;
at least one of R₁, R₂, R₃, R₄ or R₅, on at least one of W1, W2 and the or each W is selected from the group;
alkylphosphines of the formula 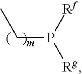
alkyl amines of the formula 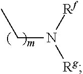
wherein each of R^{f} and R^{g} is independently selected from; 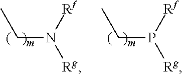
H, C₁-C₈ alkyl; and m is 1-8;
and wherein;
each of the remaining R₁, R₂, R₃, R₄ or R₅ is independently selected from;
halogen selected from the group F, Cl, Br, I; C₁₋₈ alkyl; carboxylic acid of the formula —C(O)OH; alkyl carboxylic acid of the formula —(CH₂)ₙC(O)OH; thiocarboxylic acid of the formula —C(S)OH; Alkyl thiocarboxylic acid of the formula —(CH₂)ₙC(S)OH; ester of the formula —C(O)OR^{a}; alkyl ester of the formula —(CH₂)ₙC(O)OR^{a}; Thioester of the formula —C(S)OR^{a}; alkyl thioester of the formula —(CH₂)ₙC(S)OR^{a}; dithioester of the formula —C(S)SR^{a}; alkyl dithioester of the formula —(CH₂)ₙC(S)SR^{a}; amide of the formula —C(O)NR^{a}R^{b}; alkyl amide of the formula —(CH₂)ₙC(O)NR^{a}R^{b}; thioamide of the formula —C(S)NR^{a}R^{b}; alkyl thioamide of the formula —(CH₂)ₙC(S)NR^{a}R^{b}; aldehyde of the formula —C(O)H; alkyl aldehyde of the formula —(CH₂)ₙC(O)H; thial of the formula —C(S)H; alkyl thial of the formula —(CH₂)ₙC(S)H; ketone of the formula —C(O)R^{a}; alkyl ketone of the formula —(CH₂)ₙC(O) R^{a}; thioketone of the formula —C(S)R^{a}; alkyl thioketone of the formula —(CH₂)nC(S)R^{a}; acetal, of one of the formulae: 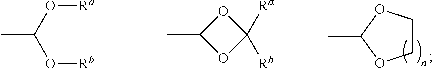
alkyl acetal, of one of the formulae: 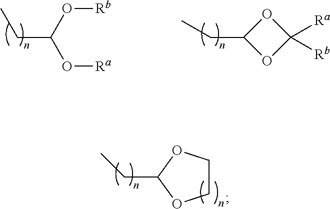
alkyl dithioacetals, of one of the formulae: 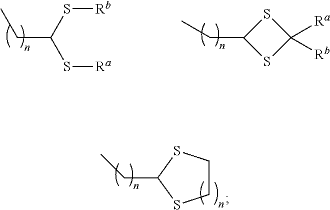
amine of the formula —NR^{a}R^{b}; alkyl amine of the formula —(CH₂)ₙNR^{a}R^{b}; ammonium salt of the formula —N⁺R^{a}R^{b}R^{c} G⁻; alkyl ammonium salt of the formula —(CH₂)ₙN⁺R^{a}R^{b}R^{c} G⁻; amide of the formula —NR^{a}C(O)R^{b}; alkyl amide of the formula —(CH₂)ₙNR^{a}C(O)R^{b}; thioamide of the formula —NR^{a}C(S)R^{b}; alkyl thioamide of one of the formulae: 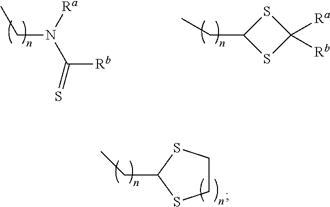
imine of one of the formulae: 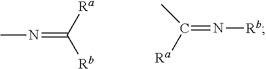
alkyl imine, of one of the formulae: 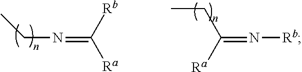
guanidine of the formula 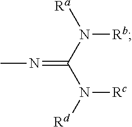
alkyl guanidine of the formula 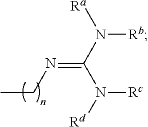
amidine of the formula 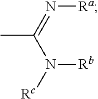
alkyl amidine of the formula 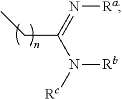
nitrile (cyano) of the formula —CN; alkyl nitrile of the formula —(CH₂)ₙCN; isonitrile of the formula —N⁺C⁻; alkyl isonitrile of the formula —(CH₂)ₙN⁺C⁻; cyanate of the formula —OCN; alkyl cyanate of the formula —(CH₂)ₙOCN; isocyanate of the formula N═C═O; alkyl isocyanate of the formula —(CH₂)ₙN═C═O; thiocyanate of the formula —SCN; alkyl thiocyanate of the formula —(CH₂)ₙ SCN; isothiocyanate of the formula —N═C═S; alkylisothiocyanate of the formula —(CH₂)ₙN═C═S; azo of the formula —N═NH; alkyl azo of the formula —(CH₂)ₙN═NH; nitro of the formula —NO₂; alkyl nitro of the formula —(CH₂)ₙNO₂; nitrite of the formula —O—N═O; alkyl nitrite of the formula —(CH₂)ₙO—N═O; nitriso of the formula —N═O; alkyl nitriso of the formula —(CH₂)ₙN═O; N-terminal peptide sequences of the formula 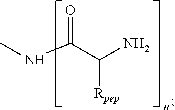
C-terminal peptide sequences of the formula 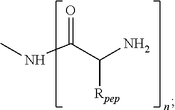
N-terminal peptide alkyl sequence of the formula 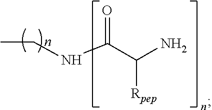
C-terminal peptide alkyl sequence of the formula 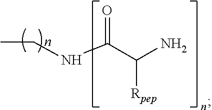
phosphine of the formula —PR^{a}R^{b}; alkyl phosphine of the formula —(CH₂)ₙPR^{a}R^{b}; phosphonium salt of the formula —P⁺R^{a}R^{b}R^{c} G⁻; alkyl phosphonium salt of the formula —(CH₂)ₙP⁺R^{a}R^{b}R^{c} G⁻; phosphine oxide of the formula —P(O)R^{a}R^{b}; alkylphosphine oxide of the formula —(CH₂)ₙP(O)R^{a}R^{b}; phosphites of the formula —O—P(OR^{a})OR^{b}; alkyl phosphites of the formula —(CH₂)ₙO—P(OR^{a})OR^{b}; phosphate of the formula —OP(O)(OR^{a})(OR^{b}); alkyl phosphate of the formula —(CH₂)ₙOP(O)(OR^{a})(OR^{b}); phosphinites of one of the formulae —OPR^{a}R^{b} or —P(R^{a})OR^{b}; alkyl phosphinites of one of the formulae —(CH₂)ₙOPR^{a}R^{b} or —(CH₂)ₙ P(R^{a})OR^{b}; phosphinates of one of the formulae —OP(O)(R^{a})(R^{b}) or —P(O)(R^{a})(OR^{b}); alkyl phosphinates of one of the formulae —(CH₂)ₙOP(O)(R^{a})(R^{b}) or —(CH₂)ₙP(O)(R^{a})(OR^{b}); phosphinites of one of the formulae —OP(OR^{a})(R^{b}) or —P(OR^{a})(OR^{b}); alkyl phosphinites of one of the formulae —(CH₂)ₙOP(OR^{a})(R^{b}) or —(CH₂)ₙP(OR^{a})(OR^{b}); phosphonates of one of the formulae —OP(O)(OR^{a})(R^{b}) or —P(O)(OR^{a})(OR³); alkyl phosphonates of one of the formulae —(CH₂)ₙOP(O)(OR^{a})(R^{b}) or —(CH₂)ₙP(O)(OR^{a})(OR^{b}); sulfate of the formula —S(O)(O)R^{a}; alkyl sulfate of the formula —(CH₂)ₙS(O)(O)R^{a}; sulfone of the formula —S(O)(O)R^{a}; alkyl sulfone of the formula —(CH₂)ₙS(O)(O)R^{a}; sulfoxide of the formula —S(O)R^{a}; alkyl sulfoxide of the formula —(CH₂)ₙS(O)R^{a}; sulfinic acid of the formula —S(O)OR^{a}; alkyl sulfinic acid of the formula —(CH₂)ₙS(O)OR^{a}; sulfimine of one of the formulae —S(R^{a})═N—R^{b} or —N═SR^{a}R^{b}; alkyl sulfimine of one of the formulae —(CH₂)ₙS(R^{a})═N—R^{b} or —(CH₂)ₙN═SR^{a}R^{b}; sulfonamide of the formula —S(O)(O)NR^{a}R¹³; alkyl sulfonamide of the formula —(CH₂)ₙS(O)(O)NR^{a}R^{b}; boronic acid of the formula —B(OH)₂; alklyl boronic acid of the formula —(CH₂)ₙB(OH)₂; boronic ester of the formula —B(OR^{a})(OR^{b}); alkyl boronic ester of the formula —(CH₂)ₙB(OR^{a})(OR^{b});
semicarbazones of the formula 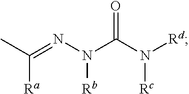
alkyl semicarbazones of the formula 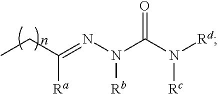
thiosemicarbazones of the formula 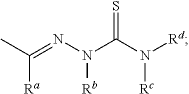
alkyl thiosemicarbazones of the formula 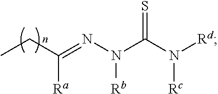
cyanimide of the formula —N(R^{a})CN; alkyl cyanimide of the formula —(CH₂)ₙN(R^{a})CN; hydrazone of the formula —C(R^{a})═N—NH₂; alkyl hydrazone of the formula —(CH₂)ₙC(R^{a})═N—NH₂; oxime of the formula —C(R^{a})═N—OH; alkyl oxime of the formula —(CH₂)ₙC(R^{a})═N—OH; nitraomine of the formula —N(R^{a})NO₂; alkyl nitraomine of the formula —(CH₂)ₙN(R^{a})NO₂; nitronate of the formula 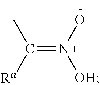
alkyl nitronate of the formula 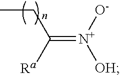
nitrone of the formula 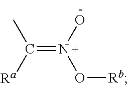
alkyl nitrone of the formula 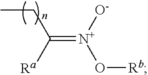
carbonate of the formula —OC(O)OR^{a}; alkyl carbonate of the formula —(CH₂)ₙOC(O)OR^{a}; carbamate of one of the formulae —OC(O)NR^{a}R^{b} or —N(R^{a})C(O)OR^{b}; alkyl carbamate of one of the formulae —(CH₂)ₙOC(O)NR^{a}R^{b} or —(CH₂)ₙN(R^{a})C(O)OR^{b}; dithiocarbamate of one or more of the formulae —SC(S)NR^{a}R^{b} or —N(R^{a})C(S)SR^{b}; alkyl dithiocarbamate of one or more of the formulae —(CH₂)ₙSC(S)NR^{a}R^{b} or —(CH₂)ₙN(R^{a})C(S)SR^{b}; substituted methane of the formula —CR^{r}R^{s}R^{t}; alkyl substituted methane of the formula —(CH₂)ₙCR^{r}R^{s}R^{t}; hydroxyl of the formula —OH; alkylhydroxyl of the formula —(CH₂)ₙOH; ether of the formula —OR^{a}; alkyl ether of the formula —(CH₂)ₙOR^{a}; amide of the formula —NH—C(O)—R^{b}; alkyl amide of the formula —(CH₂)ₙNH—C(O)—R^{b}; ester of the formula —OC(O)R^{b}; alkyl ester of the formula —(CH₂)ₙOC(O)R^{b}; phenyl; alkyl phenyl of the formula —(CH₂)ₙPh; vinyl of the formula —C═C(R^{a})(R^{b}); alkyl vinyl of the formula —(CH₂)ₙC═C(R^{a})(R^{b}); a 5- or 6-membered saturated heterocyclic ring containing 1 to 3 heteroatoms chosen independently from nitrogen, sulfur and oxygen, a 5- or 6-membered saturated heteroaromatic ring containing 1 to 5 heteroatoms chosen independently from nitrogen, sulfur and oxygen, a 5-membered unsaturated heterocyclic ring containing, one or two double bonds and containing 1 to 5 heteroatoms chosen independently from nitrogen, sulphur and oxygen, a 6 membered ring containing 1 to 3 double bonds and containing 1 to 5 heteroatoms chosen independently from nitrogen, sulfur and oxygen dependant on ring size;
wherein R^{a}, R^{b}, R^{c} and R^{d} are independently selected from; H or C₁₋₄ alkyl, Rₚₑₚ is any group resulting in the formation of an amino acid; X is a halogen; and n=1-4; and R^{r}, R^{s} and R^{t} are independently selected from H, C₁₋₄ alkyl or a halogen, with at least one of R^{r}, R^{r} or R^{t} being a halogen; G is an anion;
and wherein, on any one of W1, W2 or W where none of R₁, R₂, R₃, R₄ or R₅, is selected from the group; alkylphosphines of the formula 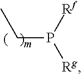
alkyl amines of the formula 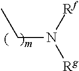
wherein each of R^{f} and R^{g} is independently selected from the group: 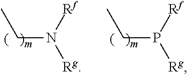
H or C₁-C₈ alkyl; and m is 1-8; none of R₁, R₂, R₃, R₄ or R₅ may be selected from the group;
halogen selected from the group F, Cl, Br, I; alkyl halogen of the formula —(CH₂)ₙ—X; carboxylic acid of the formula —C(O)OH, alkyl carboxylic acid of the formula —(CH₂)ₙC(O)OH; ester of the formula —OC(O)R^{b}; alkyl ester of the formula —(CH₂)ₙOC(O)R^{b}; aldehyde of the formula —C(O)H; alkyl aldehyde of the formula —(CH₂)ₙC(O)H; ketone of the formula —(O)R^{a}; alkyl ketone of the formula —(CH₂)ₙC(O) R^{a}; sulfate of the formula —S(O)(O)R^{a}; alkyl sulfate of the formula —(CH₂)ₙS(O)(O)R^{a}; nitrile (cyano) of the formula —CN; alkyl nitrile of the formula —(CH₂)ₙCN; nitro of the formula —NO₂; alkyl nitro of the formula —(CH₂)ₙNO₂; amide of the formula —C(O)NR^{a}R^{b}; alkyl amide of the formula —(CH₂)ₙC(O)NR^{a}R^{b}; amide of the formula —NR^{a}C(O)R^{b}; alkyl amide of the formula —(CH₂)ₙNR^{a}C(O)R^{b};
and wherein R^{a}, R^{b}, R^{c} and R^{d} are independently selected from H or C₁₋₄ alkyl; X is a halogen; and n=1-4;
or a pharmaceutically acceptable salt thereof.

2. A compound of Formula (I) according to claim 1 wherein at least one of R₁, R₂, R₃, R₄ or R₅, on each of W1, W2 and the or each W is independently selected from the group;
alkylphosphines of the formula 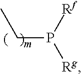
alkyl amines of the formula 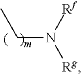
wherein each of R^{f} and R^{g} is independently selected from the group: 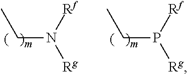
H, C₁-C₈ alkyl; and m is 1-8.

3. A compound of Formula (I) according to any one of the preceding claims wherein 2 or more of R₁, R₂, R₃, R₄ or R₅, on at least one of W1, W2 and the or each W is independently selected from the group;
alkylphosphines of the formula 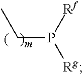
alkyl amines of the formula 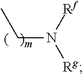
wherein each of R^{f} and R^{g} is independently selected from the group: 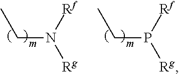
H, C₁-C₈ alkyl; and m is 1-8.

4. A compound of Formula (I) according to any one of the preceding claims, characterised in that none of R₁, R₂, R₃, R₄ or R₅ is an electron withdrawing group located in the ortho or para positions relative to the or each of R₁, R₂, R₃, R₄ or R₅ selected from the group;
alkylphosphines of the formula 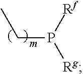
alkyl amines of the formula 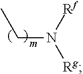
wherein each of R^{f} and R^{g} is independently selected from the group: 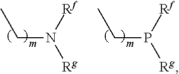
H, C₁-C₈ alkyl; and m is 1-8.

5. A compound of Formula (I) according to any one of the preceding claims, characterised in that none of R₁, R₂, R₃, R₄ or R₅ is an electron donating group located in the meta position relative to the or each of R₁, R₂, R₃, R₄ or R₅ selected from the group;
alkylphosphines of the formula 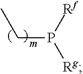
alkyl amines of the formula 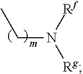
wherein each of R^{f} and R^{g} is independently selected from the group: 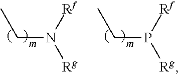
H, C₁-C₈ alkyl; and m is 1-8.

6. A compound of Formula (I) according to any one of the preceding claims, characterised in that at least one of R₁, R₂, R₃, R₄ or R₅ is an electron donating group located in the ortho or para positions relative to the or each of R₁, R₂, R₃, R₄ or R₅ selected from the group;
alkylphosphines of the formula 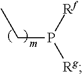
alkyl amines of the formula 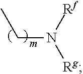
wherein each of R^{f} and R^{g} is independently selected from the group: 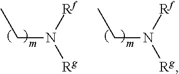
H, C₁-C₈ alkyl; and m is 1-8.

7. A compound of Formula (I) according to any one of the preceding claims, characterised in that at least one of R₁, R₂, R₃, R₄ or R₅ is an electron withdrawing group located in the meta position relative to the or each of R₁, R₂, R₃, R₄ or R₅ selected from the group;
alkylphosphines of the formula 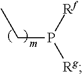
alkyl amines of the formula 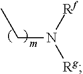
wherein each of R^{f} and R^{g} is independently selected from the group: 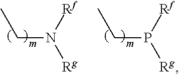
H, C₁-C₈ alkyl; and m is 1-8.

8. A compound of Formula (I) according to claim 4 or 7 wherein the electron withdrawing group is selected from the following group:
halogen selected from the group F, Cl, Br, I; alkyl halogen of the formula —(CH₂)ₙ—X; carboxylic acid of the formula —C(O)OH, alkyl carboxylic acid of the formula —(CH₂)ₙC(O)OH; ester of the formula —OC(O)R^{b}; alkyl ester of the formula —(CH₂)ₙOC(O)R^{b}; aldehyde of the formula —C(O)H; alkyl aldehyde of the formula —(CH₂)ₙC(O)H; ketone of the formula —C(O)R^{a}; alkyl ketone of the formula —(CH₂)ₙC(O) R^{a}; sulfate of the formula —S(O)(O)R^{a}; alkyl sulfate of the formula —(CH₂)ₙS(O)(O)R^{a}; nitrile (cyano) of the formula —CN; alkyl nitrile of the formula —(CH₂)ₙCN; nitro of the formula —NO₂; alkyl nitro of the formula —(CH₂)ₙNO₂; amide of the formula —C(O)NR^{a}R^{b}; alkyl amide of the formula —(CH₂)ₙC(O)NR^{a}R^{b}; amide of the formula —NR^{a}C(O)R^{b}; alkyl amide of the formula —(CH₂)ₙNR^{a}C(O)R^{b};
wherein R^{a}, R^{b}, R^{c} and R^{d} are independently selected from H or C₁₋₄ alkyl and where X is a halogen and n=1-4.

9. A compound of Formula (I) according to claim 5 or 6 wherein the electron donating group is selected from the following group:
amine of the formula —NR^{a}R^{b}; alkyl amine of the formula —(CH₂)ₙNR^{a}R^{b}; phosphine of the formula —PR^{a}R^{b}; alkyl phosphine of the formula —(CH₂)ₙPR^{a}R^{b}; hydroxyl of the formula —OH; alkylhydroxyl of the formula —(CH₂)ₙOH; ether of the formula —OR^{a}; alkyl ether of the formula —(CH₂)ₙOR^{a}; amide of the formula —NH—C(O)—R^{b}; alkyl amide of the formula —(CH₂)ₙNH—C(O)—R^{b}; ester of the formula —OC(O)R^{b}; alkyl ester of the formula —(CH₂)ₙOC(O)R^{b}; phenyl; alkyl phenyl of the formula —(CH₂)ₙPh; vinyl of the formula —C═C(R^{a})(R^{b}); alkyl vinyl of the formula —(CH₂)ₙC═C(R^{a})(R^{b}); guanidines of the formula 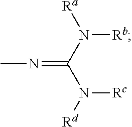
alkyl guanidines of the formula 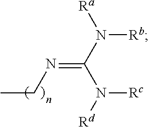
amidine of the formula 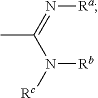
and alkyl amidine of the formula 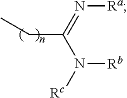
wherein R^{a}, R^{b}, R^{c} and R^{d} are independently selected from H or C₁₋₄ alkyl and where X is a halogen and n=1-4.

10. A compound of Formula (I) according to any one of the preceding claims, wherein each of W1, W2 and the, or each, W is identical.

11. A compound of Formula (I) according to any one of the preceding claims, wherein each of W1, W2 and the, or each, W is independently selected from the group: C₂₋₄ alkyl groups; C₂ E-alkene, with V and Z in the 1 and 2 positions, and C₂ Z-alkene, with V and Z in the 1 and 2 positions.

12. A compound of Formula (I) according to any one of the preceding claims, wherein each of W1, W2 and the, or each, W is independently selected from the group:; C₂ E-alkene, with V and Z in the 1 and 2 positions, and C₂ Z-alkene, with V and Z in the 1 and 2 positions.

13. A compound of Formula (I) according to any one of the preceding claims wherein at least one of R₁, R₂, R₃, R₄ or R₅, on at least one of W1, W2 and the or each W is selected from the group;
alkyl amines of the formula 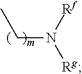
wherein each of R^{f} and R^{g} is independently selected from the group: 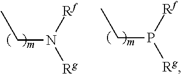
H, C₁-C₈ alkyl; and m is 1-8.

14. A compound of Formula (I) according to any one of the preceding claims wherein at least one of R₁, R₂, R₃, R₄ or R₅, on at least one of W1, W2 and the or each W is selected from the group;
alkyl amines of the formula 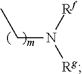
wherein each of R^{f} and R^{g} is independently selected from the group: H, C₁-C₈ alkyl; and m is 1-8.

15. A compound of Formula (I) according to any one of the preceding claims wherein at least one of R₁, R₂, R₃, R₄ or R₅, on at least one of W1, W2 and the or each W is selected from the group;
alkyl amines of the formula 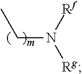
wherein each of R^{f} and R^{g} is independently selected from the group: H, C₁-C₄ alkyl.

16. A compound of Formula (I) according to any one of the preceding claims wherein at least one of R₁, R₂, R₃, R₄ or R₅, on at least one of W1, W2 and the or each W is selected from the group;
alkyl amines of the formula 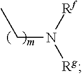
wherein each of R^{f} and R^{g} is independently selected from the group: H, C₁-C₂ alkyl.

17. A compound of Formula (I) according to any one of the preceding claims wherein at least one of R₁, R₂, R₃, R₄ or R₅, on at least one of W1, W2 and the or each W is selected from the group;
alkyl amines of the formula 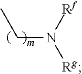
wherein m is 1-4.

18. A compound of Formula (I) according to any one of the preceding claims wherein at least one of R₁, R₂, R₃, R₄ or R₅, on at least one of W1, W2 and the or each W is selected from the group;
alkyl amines of the formula 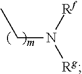
wherein m is 1.

19. A compound of Formula (I) according to any one of the preceding claims wherein V is a phenyl ring.

20. A compound of Formula (I) according to any one of the preceding claims wherein each Z is a phenyl ring.

21. A compound of Formula (I) having the following structural formula: 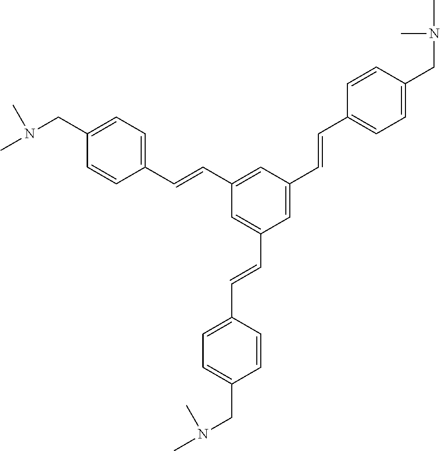
or pharmaceutically acceptable salts thereof.

22. A compound of Formula (I) having the following structural formula: 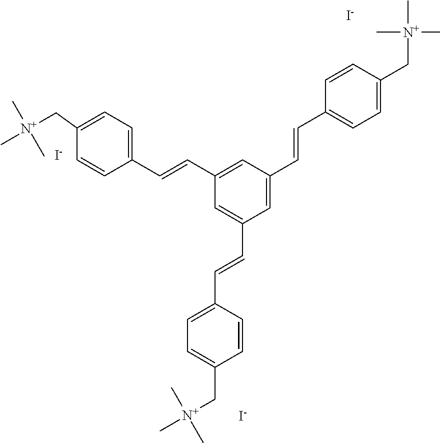
or pharmaceutically acceptable salts thereof.

23. A method of treating a Gram-negative bacterial infection in a subject comprising the step of administering to the subject an effective amount of a compound of Formula (I) according to claim 1 or pharmaceutically acceptable salts thereof.

24. The method of claim 23 wherein none of R₁ to R₅ of a compound of Formula (I) according to claim 1 or pharmaceutically acceptable salts thereof are:
the ammonium salt of the formula 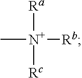
the alkyl ammonium salt of the formula 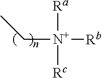
the phosphonium salt of the formula 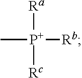
or the alkyl phosponium salt of the formula

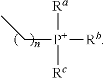

25. The use of a compound of Formula (I) according to claim 1 or pharmaceutically acceptable salts thereof, in the manufacture of a medicament for the treatment or prevention of a bacterial infection or disease in a subject in need thereof, wherein the bacterial infection or disease results from a Gram-negative bacteria.

26. A formulation comprising a therapeutically-effective amount of a compound of Formula (I) according to claim 1 and pharmaceutically acceptable salts thereof, and a pharmaceutically acceptable carrier or diluent.

27. A compound of Formula (I) according to claim 1 or pharmaceutically acceptable salts thereof, for use in a method of treating or preventing Gram-negative bacterial infections in a subject in need thereof, the method including the step of administering a therapeutically effective amount of a compound of Formula (I), or a therapeutically acceptable salt thereof, to the subject, wherein the bacterial infection or disease results from a Gram-negative bacteria.

28. The use of a compound of Formula (I) according to claim 1 or pharmaceutically acceptable salts thereof, for the treatment or prevention of a bacterial infection or disease in a subject in need thereof, wherein the bacterial infection or disease results from a Gram-negative bacteria.
